(19) United States
(12) Patent Application Publication
(10) Pub. No.: US 2023/0414555 A1
(43) Pub. Date: Dec. 28, 2023

(54) USE OF DIHYDROMYRICETIN AS EPIGENETIC ACTIVE INGREDIENT FOR COSMETIC OR DERMATOLOGICAL SKIN CARE

(71) Applicant: BEIERSDORF AG, Hamburg (DE)

(72) Inventors: Marc WINNEFELD, Wedel (DE); Elke Hendrik GROENNIGER, Hamburg (DE); Boris KRISTOF, Halstenbek (DE); Cassandra FALCKENHAYN, Hamburg (DE); Nicholas HOLZSCHECK, Hamburg (DE); Joern SOEHLE, Bremen (DE); Sascha BAUMANN, Grossenrade (DE)

(21) Appl. No.: 18/035,346

(22) PCT Filed: Sep. 27, 2021
(86) PCT No.: PCT/EP2021/076510

§ 371 Date: May 4, 2023

(30) Foreign Application Priority Data
Nov. 6, 2020 (DE) 10 2020 213 959.8

(51) Int. Cl. A61K 31/352; A61K 8/49; A61K 9/00; A61K 45/06; A61K 9/107; A61K 8/06; A61Q 19/08; A61P 17/00

(52) U.S. Cl. CPC A61K 31/352; A61K 8/498; A61K 9/0014; A61K 45/06; A61K 9/107; A61K 8/06; A61Q 19/08; A61P 17/00

(57) ABSTRACT

Use of dihydromyricetin as an epigenetic active ingredient for cosmetic or dermatological skin care.

The present invention relates to cosmetic or dermatological preparations containing active ingredients for the care and protection of the skin, in particular sensitive skin and also, very especially, skin which is aging or has aged as a result of intrinsic and/or extrinsic factors, and to the use of such active ingredients and combinations of such active ingredients in the field of cosmetic and dermatological skin care.

In a more specific sense, the invention relates to the use of such active ingredients and combinations of such active ingredients as epigenetic active ingredients, in particular to the use thereof for the partial or complete restoration of the youthful epigenome.

Epigenetic processes are all those in a cell which are considered to be “additional” to the contents and operations of genetics. In 1942, when the structure of DNA was still unknown, epigenetics was (first) defined as “the branch of biology which studies the causal interactions between genes and their products which bring the phenotype into being”.

In order to delineate from the general concept of gene regulation, today's definitions are usually more specific, for example: “The term ‘epigenetic’ defines all meiotically and mitotically heritable changes in gene expression which are not themselves encoded in the DNA sequence”.

Epigenetic processes can involve blocking the expression of a gene. The blocked gene is not lost in cell division or inheritance, but merely inactive and can be reactivated with the aid of suitable aids.

Cosmetic skin care is primarily to be understood as meaning the strengthening or restoring of the skin's natural function as a barrier against environmental influences (for example dirt, chemicals, microorganisms) and against the loss of endogenous substances (for example water, natural fats, electrolytes).

Impairment of this function can lead to increased absorption of toxic or allergenic substances or colonization by microorganisms, resulting in toxic or allergic skin reactions.

A further aim of skin care is to compensate for the loss of fats and water from the skin caused by daily washing. This becomes particularly important if the natural capacity for regeneration is inadequate. In addition, skin care products should protect against environmental influences, in particular against sun and wind, and slow the aging of the skin.

Chronological aging of the skin is caused for example by endogenous, genetically determined factors. In the epidermis and dermis, aging results for example in the following structural damage and functional impairments, which can also be grouped together under the term “senile xerosis”:
a) dryness, roughness and formation of fine wrinkles caused by dryness,
b) itching
   and
c) decreased refatting by sebaceous glands (for example after washing).

Exogenous factors, such as UV light and chemical noxae, can have a cumulative effect and for example accelerate the endogenous aging processes or add to them. In the epidermis and dermis, exogenous factors in particular result for example in the following structural damage and functional impairments in the skin, which go beyond the damage from chronological aging in both extent and quality:
d) visible vasodilation phenomena (telangiectasias, couperose);
e) sagging skin and formation of wrinkles;
f) local hyper-, hypo- and dyspigmentation (for example age spots) and
g) increased susceptibility to mechanical stress (for example cracking).

The present invention relates in particular to products for the care of naturally aged skin, and for the treatment of the consequential damage of photoaging, in particular the phenomena listed under a) to g).

Products for the care of aged skin are known per se. They contain, for example, retinoids (vitamin A acid and/or derivatives thereof) or vitamin A and/or derivatives thereof. However, their effect on structural damage is of limited extent. Moreover, in product development there are considerable difficulties with sufficiently stabilizing the active ingredients against oxidative degradation. The use of products containing vitamin A acid often, moreover, causes severe erythematous skin irritation. Retinoids can therefore be used only in low concentrations.

Epigenetics is still a relatively new field of research in life sciences that has attracted a lot of attention in recent years. This is not surprising since epigenetics examines how environmental factors affect our bodies. It is less about the further descriptive analysis of the morphological changes described above, and more about the understanding of the underlying molecular-biological regulation mechanisms. In contrast to classical genetics, epigenetics is concerned not with changes in the primary sequence of DNA, but rather with the mechanisms of gene regulation. Epigenetics acts as a link between the environment and the genome.

One of the best described components of epigenetics is what is known as DNA methylation. DNA methylation is a modification of DNA which, in mammals, generally takes place symmetrically on both DNA strands at the C5 position of cytosine nucleotides when these are located next to guanosine nucleotides in the 5′ direction (CpG) [Bird, 202]. The high mutation potential of the methylated cytosine nucleotides means that the proportion of CpG dinucleotides in relation to the entire genome is very low. The hydrolytic deamination of the methylated cytosine spontaneously leads to the formation of thymine, resulting in a TG misbase pairing. In comparison thereto, the deamination of an unmethylated cytosine to form uracil takes place much more slowly and the repair of the resulting UG misbase pairing is much more efficient since uracil is not a naturally occurring base in DNA [Coulondre et al., 1978; Jurkowska et al., 2010].

Despite the high mutation rate, there are a few CG-rich sections of DNA in the genome, referred to as CpG islands. These short sections of DNA are defined as regions that are 0.5-4 kb long and have a ratio of actual to expected CG content of greater than 0.65 [Takai and Jones, 2002]. About 70% of all promoters of the human genome are associated with such CpG islands [Saxonov et al., 2006]. There they are generally in unmethylated form, this correlating with the transcriptional expression of the corresponding gene. However, some biological processes are also known in which the methylation of a CpG island results in the silencing of specific genes. Examples of these include the inactivation of the X chromosome and germline- and tissue-specific genes [Bird, 2002, Avner and Heard, 2001, Bird, 1986]. This illustrates the fundamental role of DNA methylation for the regulation of gene expression and the determination of cell identity.

Much more frequently, however, methylation can be found in regions remote from the promoter, such as in repetitive sequences or in parasitic sequences, where the methylation prevents the transcription of these sequences and thus contributes to the integrity of the genome [Yoder et al., 1997, Walsh et al., 1998]. Overall, about 3-5% of the cytosines in the genomic DNA are methylated, this ultimately meaning that about 80% of all the CpG loci in the genome are methylated [Ehrlich et al., 1982, Gama-Sosa et al., 1983].

In comparison to DNA methylation in healthy cells, a large number of study results have proved that the DNA methylation pattern in degenerated cancer cells is often changed. For instance, what has been increasingly found in cancer tissue is a global decrease in methylation of the genome (hypomethylation) that is primarily caused by a reduction in the level of methylation in repetitive elements [Ehrlich, 2002]. This hypomethylation can cause the reactivation of these transposing elements [Yoder et al., 1997, Wash et al., 1998] and thus has a negative effect on the genomic integrity [Gaudet, 2003]. In parallel to the global hypomethylation, it was likewise possible to observe a hypermethylation of CpG islands which are associated with the promoter region of genes [Herman and Baylin, 2000, Jones and Baylin, 2007]. This hypermethylation is often accompanied by transcriptional inactivation of the gene.

Since, inter alia, tumor suppressor genes are affected by this hypermethylation, misregulation of important cellular mechanisms occurs. In order to distinguish such epigenetic changes from classical genetic mutations, they are referred to as “epimutations” [Jeggo and Holliday, 1986].

Beyond cancer research, it has also become increasingly clear in recent years that epigenetics is just as important for the development of a healthy organism as DNA itself. Scientific studies have also made it clear that the epigenome—all epigenetic modifications as a whole—can be changed by external influences much more easily than the genes themselves. Epigenetically active molecules act as the mediators between the environment and the genetic material. It has clearly been demonstrated that external factors can switch genes on and off and thus trigger phenotypic changes and/or diseases (Jaenisch & Bird, 2003, Bird et al. 2007; Reik, 2007; Feinberg, 2008). Recent studies have also shown that epigenetic changes—particularly in the DNA methylation pattern—also occur during aging (Fraga et al. 2005; Esteller et al. 2012; Winnefeld & Lyko 2012).

In addition, it has been shown that epigenetic changes (hypermethylations) can also be observed during skin aging (Grönniger et al., 2010), resulting in a “silencing” of skin-relevant genes.

In order to achieve an improvement in the condition of the skin, it is therefore desirable to reverse epigenetic changes—which occur either with age or in the case of certain skin conditions—in order to thus reactivate the skin-relevant genes.

However, only a limited number of substances having DNA-demethylating activity are known to date.

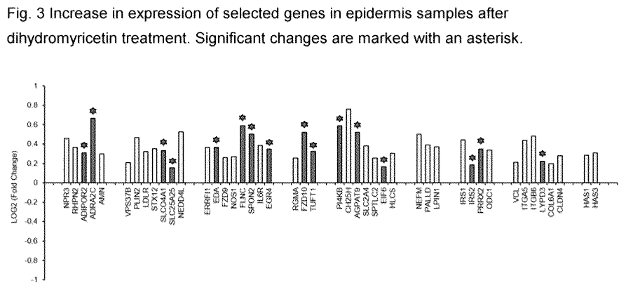

In this context, the substance dihydromyricetin surprisingly exhibited a inhibiting effect on DNA methyltransferase 1. This enzyme is responsible for methylating the CpGs in the genome and therefore for maintaining the specific methylation pattern of the cell. It was possible to demonstrate the derivable epigenetic activity of the active ingredient in the modification of the methylation pattern in skin samples after a dihydromyricetin treatment (EPIC analysis). Furthermore, the use of dihydromyricetin made it possible to achieve an increase in expression of age-dependent hypermethylated genes (shown for 16 genes, see FIG. 3).

Dihydromyricetin (or (+)-dihydromyricetin; ampelopsin; (2R,3R)-3,5,7-trihydroxy-2-(3,4,5-trihydroxyphenyl)chroman-4-one) is characterized by the following structure:

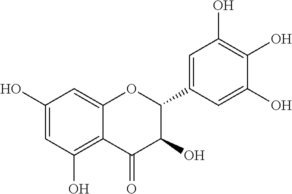 [SMILES: O=C1c2c(O)cc(O)cc2O[C@H](c2cc(O)c(O)c(O)c2)[C@H]1O]

JP 63316711 discloses the use of dihydromyricetin as a skin-lightening active ingredient. Furthermore, FR 2868701 discloses the use of dihydromyricetin as an active ingredient against cellulite, since it influences the storage of fat in fat cells. In addition, DE 10 2009 055 916 describes the use of dihydromyricetin for increasing dermal collagen synthesis, and for the treatment and prophylaxis of the symptoms of intrinsic and/or extrinsic skin aging.

Nevertheless, the prior art was unable to point the way to the present invention.

The solution to the objects on which the invention was based is consequently the use of dihydromyricetin as a demethylating agent.

This use is advantageously implemented in topical preparations, particularly cosmetic or dermatological preparations.

Cosmetic or dermatological preparations according to the invention preferably contain 0.001-10% by weight, particularly preferably 0.01-1% by weight, of dihydromyricetin, based on the total composition of the preparations.

Emulsions are advantageous presentation forms in the context of the present invention, for example are advantageous in the form of a cream, a lotion, a cosmetic milk, and contain for example fats, oils, waxes and/or other fatty substances, and water and one or more emulsifiers, as are customarily used for such a type of formulation.

Medicinal topical compositions in the context of the present invention generally contain one or more medicaments at an effective concentration. For the sake of simplicity, in order to make a clear distinction between cosmetic and medicinal use and corresponding products, reference is made to the statutory provisions of the Federal Republic of Germany (for example the Kosmetikverordnung [Cosmetics Ordinance] and Lebensmittel-und Arzneimittelgesetz [Food and Medicinal Products Act]).

It is also advantageous here for the active ingredient used according to the invention to be included as an additive in preparations that already contain other active ingredients for other purposes.

If the cosmetic or dermatological preparation in the context of the present invention is a solution or emulsion or dispersion, it is possible to use, as solvents:
water or aqueous solutions
oils, such as capric or caprylic acid triglycerides, but preferably castor oil;
fats, waxes and other natural and synthetic fatty substances, preferably esters of fatty acids with alcohols of low carbon number, for example with isopropanol, propylene glycol or glycerol, or esters of fatty alcohols with alkanoic acids of low carbon number or with fatty acids;
alcohols, diols or polyols of low carbon number, and the ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

The oil phase of the emulsions, oleogels or hydrodispersions or lipodispersions in the context of the present invention is advantageously selected from the group of the esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, from the group of the esters of aromatic carboxylic acids and saturated and/or unsaturated, branched and/or unbranched alcohols having a chain length of 3 to 30 carbon atoms. Such ester oils may then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, for example jojoba oil.

In addition, the oil phase may advantageously be selected from the group of branched and unbranched hydrocarbons and hydrocarbon waxes, silicone oils, dialkyl ethers, the group of saturated or unsaturated, branched or unbranched alcohols, and fatty acid triglycerides, namely the triglyceryl esters of saturated and/or unsaturated, branched and/or unbranched alkanecarboxylic acids having a chain length of 8 to 24, in particular 12-18, carbon atoms. The fatty acid triglycerides may for example advantageously be selected from the group of synthetic, semisynthetic and natural oils, for example olive oil, sunflower oil, soybean oil, groundnut oil, rapeseed oil, almond oil, palm oil, coconut oil, palm kernel oil, and other similar oils.

Any desired blends of such oil and wax components are also advantageous in the

The aqueous phase of the preparations according to the invention may advantageously contain
alcohols, diols or polyols of low carbon number, and the ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low carbon number, for example ethanol, isopropanol, propane-1,2-diol, glycerol, and in particular one or more thickeners, which may advantageously be selected from the group of silicon dioxide, aluminum silicates, polysaccharides and derivatives thereof, for example hyaluronic acid, xanthan gum, hydroxypropyl methylcellulose, particularly advantageously from the group of polyacrylates, preferably a polyacrylate from the group of the so-called Carbopols, for example Carbopols of types 980, 981, 1382, 2984, 5984, each individually or in combination.

Gels used according to the invention usually contain alcohols of low carbon number, for example ethanol, isopropanol, propane-1,2-diol, glycerol, and water or an abovementioned oil in the presence of a thickener, which is preferably silicon dioxide or an aluminum silicate in the case of oily/alcoholic gels and is preferably a polyacrylate in the case of aqueous/alcoholic or alcoholic gels.

Solid sticks contain for example natural or synthetic waxes, fatty alcohols or fatty acid esters.

Customary base materials which are suitable for use as cosmetic sticks in the context of the present invention are liquid oils (for example paraffin oils, castor oil, isopropyl myristate), semi-solid constituents (for example vaseline, lanolin), solid constituents (for example beeswax, ceresin and microcrystalline waxes or ozokerite), and high-melting point waxes (for example carnauba wax, candelilla wax).

Suitable as propellant for cosmetic and/or dermatological preparations in the context of the present invention that are sprayable from aerosol containers are the readily volatile, liquefied propellants that are customary and known, for example hydrocarbons (propane, butane, isobutane), which can be used alone or in a mixture with one another. Compressed air may also advantageously be used.

The effect of dihydromyricetin according to the invention is demonstrated below:

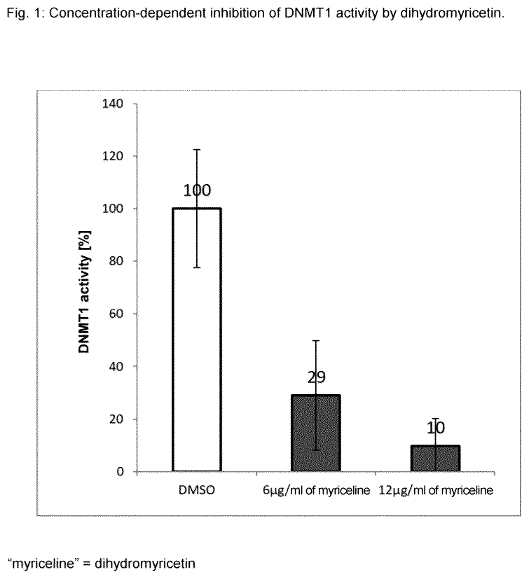

The inhibiting property of DNA methyltransferase 1 (DNMT1) by dihydromyricetin was first demonstrated in a cell-free activity assay (DNMT Activity/Inhibition Assay, Active Motif, Belgium). It was possible to show the concentration-dependent inhibition of the recombinant DNMT1 enzyme (FIG. 1). Here, the assay manufacturer's standard protocol was used.

The epigenetic modulation by dihydromyricetin in the skin was demonstrated in an in vivo study. To this end, test subjects (n=32) applied cream comprising a vehicle and a verum including dihydromyricetin to the lower back for two weeks. After the two-week treatment period, the test subjects were irradiated with UV (0.8 MED) on three consecutive days in order to imitate an age-relevant environmental influence. 24 hours after the final UV irradiation, suction blisters were produced and the epidermis was harvested. The DNA was isolated from the epidermis and the state of the DNA methylation of 850 000 CpGs was determined by means of EPIC technology from Illumina.

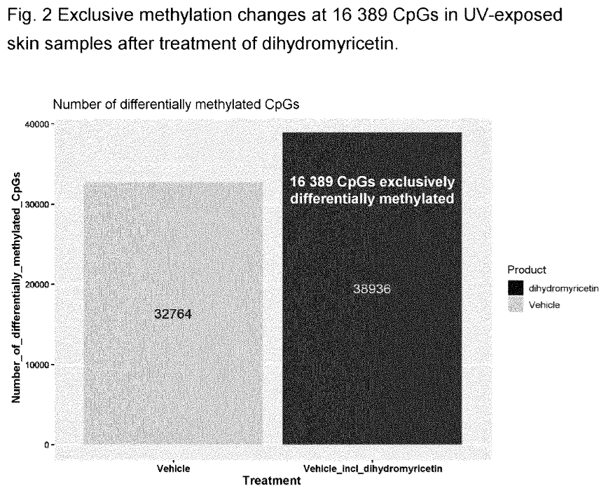

Compared to the unirradiated control, the verum-treated area exhibited methylation changes at 16 389 CpGs at which the vehicle-treated area did not show any significant methylation changes. It was thus possible to demonstrate that dihydromyricetin is an epigenetic modulator (FIG. 2).

In a further in vivo study, the reactivation of genes in the skin, which reactivation can be derived from the demethylation, was demonstrated. To this end, 19 test subjects (50-65 years) applied cream comprising a vehicle and a verum including dihydromyricetin to the inner forearm over a period of eight weeks. Suction blisters were then produced and the epidermis was harvested. The RNA was isolated from the epidermis samples (RNeasy Fibrous Tissue Kit, Qiagen, Hilden) and the expression of 45 genes (+3 endogenous control genes) was analyzed using the TaqMan system (7900HT Fast Real-Time PCR System, Applied Biosystems). For 16 genes, it was possible to demonstrate a significant increase in expression in comparison to the vehicle-treated control samples (FIG. 3). The changes in expression were calculated using the ΔΔCt method.

The example which follows is intended to illustrate the present invention. Unless stated otherwise, the numerical values relate to percentages by weight.

### EXAMPLE 1

| | |
|---|---|
| MYRICELINE | 0.15 |
| Ethylhexylglycerin | 0.3 |
| Dimethicone | 0.3 |
| Synthetic Beeswax | 3 |
| Ethylhexyl Cocoate | 3 |
| Glyceryl Stearate Citrate | 2 |
| Silica Dimethyl Silylate | 0.5 |
| VP/Hexadecene Copolymer | 0.5 |
| Glycerin | 7 |
| Aqua + Sodium Hydroxide | 0.1 |
| Phenoxyethanol + Aqua | 0.5 |
| Sodium Metabisulfite | 0.15 |
| Aqua + Trisodium EDTA | 1 |
| Alcohol Denat. + Aqua | 6 |
| Cetearyl Alcohol | 1.5 |
| Acrylates/C10-30 Alkyl | 0.25 |
| Acrylate Crosspolymer | |
| Xanthan Gum | 0.5 |
| Diethylhexyl Syringylidenemalonate + | 0.1 |
| Caprylic/Capric Triglyceride | |
| Homosalate | 4.5 |
| Octocrylene | 9 |
| Ethylhexyl Salicylate | 4.5 |
| Bis-Ethylhexyloxyphenol | 2 |
| Methoxyphenyl Triazine | |
| Butyl Methoxydibenzoylmethane | 4.5 |
| Water | To 100 |

1.-5. (canceled)

6. A method of cosmetic or dermatological skin care, wherein the method comprises topically applying to skin in need thereof dihydromyricetin as an epigenetic active ingredient.

7. The method of claim 6, wherein dihydromyricetin is applied to skin in an amount which causes DNA-demethylation.

8. The method of claim 6, wherein dihydromyricetin is applied to skin in an amount which causes a reactivation of genes that have been hypermethylated with age.

9. The method of claim 6, wherein dihydromyricetin is applied in the form of a cosmetic or dermatological preparation.

10. The method of claim 9, wherein the cosmetic or dermatological preparation comprises from 0.001% to 10% by weight of dihydromyricetin, based on a total weight of the preparation.

11. The method of claim 9, wherein the cosmetic or dermatological preparation comprises from 0.01% to 1% by weight, of dihydromyricetin, based on a total weight of the preparation.

12. The method of claim 9, wherein the cosmetic or dermatological preparation is present as an emulsion.

13. The method of claim 9, wherein the cosmetic or dermatological preparation further comprises one or more UV filters.

14. The method of claim 13, wherein the cosmetic or dermatological preparation comprises one or more of homosalate, octocrylene, ethylhexyl salicylate, bis-ethylhexyloxyphenol methoxyphenyl triazine, butyl methoxydibenzoylmethane.

15. The method of claim 9, wherein the cosmetic or dermatological preparation further comprises at least one further active ingredient which serves a purpose different from that served by dihydromyricetin.

16. The method of claim 9, wherein the cosmetic or dermatological preparation further comprises diethylhexyl syringylidenemalonate.

17. A cosmetic or dermatological preparation, wherein the preparation is formulated for topical use and comprises dihydromyricetin and one or more UV filter substances and/or one or more further active ingredients which serve a purpose different from that served by dihydromyricetin.

18. The cosmetic or dermatological preparation of claim 17, wherein the preparation comprises one or more UV filter substances.

19. The cosmetic or dermatological preparation of claim 17, wherein the preparation comprises one or more of homosalate, octocrylene, ethylhexyl salicylate, bis-ethylhexyloxyphenol methoxyphenyl triazine, butyl methoxydibenzoylmethane.

20. The cosmetic or dermatological preparation of claim 17, wherein the preparation comprises from 0.001% to 10% by weight of dihydromyricetin, based on a total weight of the preparation.

21. The cosmetic or dermatological preparation of claim 17, wherein the preparation comprises from 0.01% to 1% by weight of dihydromyricetin, based on a total weight of the preparation.

22. The cosmetic or dermatological preparation of claim 17, wherein the preparation comprises one or more further active ingredients which serve a purpose different from that served by dihydromyricetin.

23. The cosmetic or dermatological preparation of claim 17, wherein the preparation is present as an emulsion.

24. The cosmetic or dermatological preparation of claim 17, wherein the preparation comprises dihydromyricetin in a concentration which is sufficient to cause DNA-demethylation when applied to skin.

25. The cosmetic or dermatological preparation of claim 17, wherein the preparation comprises dihydromyricetin in a concentration which is sufficient to cause a reactivation of genes that have been hypermethylated with age when applied to skin.
